(19) United States
(12) Patent Application Publication
(10) Pub. No.: US 2023/0136910 A1
(43) Pub. Date: May 4, 2023

(54) Synthesis of vinylic alcohol intermediates

(71) Applicant: AMGEN INC., Thousand Oaks, CA (US)

(72) Inventors: Robert P. FARRELL, Thousand Oaks, CA (US); Jason S. TEDROW, Salem, MA (US)

(73) Assignee: AMGEN INC., Thousand Oaks, CA (US)

(21) Appl. No.: 17/916,843

(22) PCT Filed: Apr. 28, 2021
(86) PCT No.: PCT/US21/29526

§ 371 Date: Oct. 4, 2022

Related U.S. Application Data
(60) Provisional application No. 63/020,888, filed on May 6, 2020.

(51) Int. Cl. C07D 519/00; C07D 513/04

(52) U.S. Cl. CPC C07D 519/00; C07D 513/04

(57) ABSTRACT

Provided herein are processes for synthesizing intermediates useful in preparing Mcl-1 inhibitors. In particular, provided herein are processes for synthesizing compound (E), wherein R¹ is described herein. Compound (E) can be useful in synthesizing compound (A1), or a salt or solvate thereof, and compound (A2), or a salt of solvate thereof.

### CROSS REFERENCES TO RELATED APPLICATIONS

This application claims the benefit of U.S. Provisional Application No. 63/020,888, filed on May 6, 2020, which is hereby incorporated by reference in its entirety and for all purposes as if fully set forth herein.

### BACKGROUND

### Technical Field

The present disclosure relates to processes for synthesizing intermediates useful in preparing (1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}.0^{19,24}]pentacosa[8,16,18,24]tetraen]-15′-one 13′,13′-dioxide (compound A1; AMG 176), a salt, or solvate thereof, and in preparing (1S,3′R,6′R,7′R,8′E,11′S,12′R)-6-chloro-7′-methoxy-11′,12′-dinethyl-7′-((9aR)-octahydro-2H-pyrido[1,2-a]pyrazin-2-ylmethyl)-3,4-dihydro-2H,15′H-spiro[naphthalene-1,22′-[20]oxa[13]thia[1,14]diazatetracyclo [14.7.2.0^{3,6}.0^{19,24}]pentacosa[8,16,18,24]tetraen]-15′-one 13′,13′-dioxide (compound A2; AMG 397), a salt, or solvate thereof. These compounds are inhibitors of myeloid cell leukemia 1 protein (Mcl-1).

### Description of Related Technology

The compound, (1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}.0^{19,24}]pentacosa[8,16,18,24]tetraen]-15′-one 13′,13′-dioxide (compound A1), is useful as an inhibitor of myeloid cell leukemia 1 (Mcl-1):

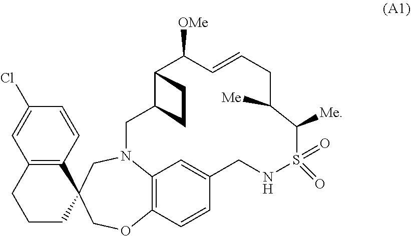

The compound, (1S,3′R,6′R,7′R,8′E,11′S,12′R)-6-chloro-7′-methoxy-11′,12′-dinethyl-7′-((9aR)-octahydro-2H-pyrido[1,2-a]pyrazin-2-ylmethyl)-3,4-dihydro-2H,15′H-spiro[naphthalene-1,22′-[20]oxa[13]thia[1,14]diazatetracyclo [14.7.2.0^{3,6}.0^{19,24}]pentacosa[8,16,18,24]tetraen]-15′-one 13′,13′-dioxide (compound A2), is useful as an inhibitor of myeloid cell leukemia 1 (Mcl-1):

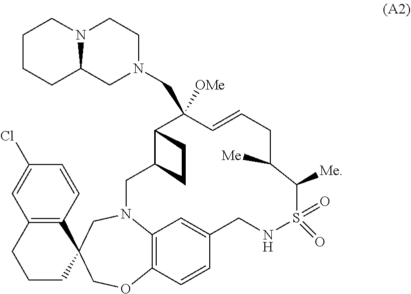

One common characteristic of human cancer is overexpression of Mcl-1. Mcl-1 overexpression prevents cancer cells from undergoing programmed cell death (apoptosis), allowing the cells to survive despite widespread genetic damage.

Mcl-1 is a member of the Bcl-2 family of proteins. The Bcl-2 family includes pro-apoptotic members (such as BAX and BAK) which, upon activation, form a homo-oligomer in the outer mitochondrial membrane that leads to pore formation and the escape of mitochondrial contents, a step in triggering apoptosis. Antiapoptotic members of the Bcl-2 family (such as Bcl-2, Bcl-XL, and Mcl-1) block the activity of BAX and BAK. Other proteins (such as BID, BIM, BIK, and BAD) exhibit additional regulatory functions. Research has shown that Mcl-1 inhibitors can be useful for the treatment of cancers. Mcl-1 is overexpressed in numerous cancers.

U.S. Pat. No. 9,562,061, which is incorporated herein by reference in its entirety, discloses compound A1 as an Mcl-1 inhibitor and provides a method for preparing it. However, improved synthetic methods that result in greater yield and purity of compound A1 are desired, particularly for the commercial production of compound A.

U.S. Pat. No. 10,300,075, which is incorporated herein by reference in its entirety, discloses compound A2 as an Mcl-1 inhibitor and provides a method for preparing it. However, improved synthetic methods that result in greater yield and purity of compound A2 are desired, particularly for the commercial production of compound A2.

### SUMMARY

Provided herein are processes for synthesizing compound E, or a salt or solvate thereof:

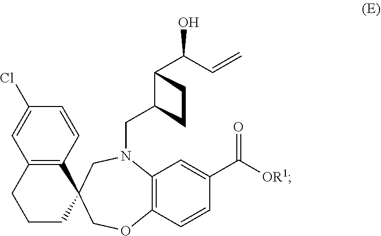

comprising admixing compound C, compound D,

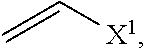

and Zn(X³)₂ in an organic solvent to form compound E:

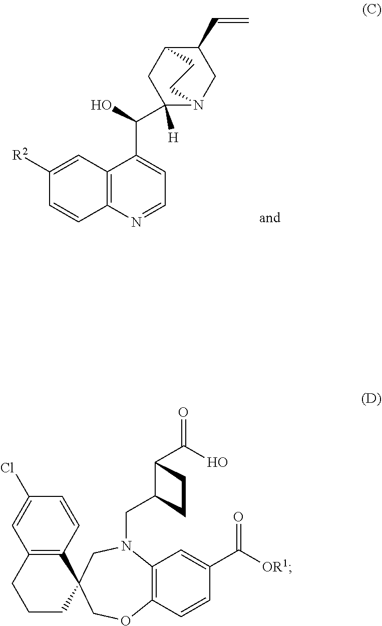

wherein R¹ is C₁₋₆alkyl; R² is H or C₁₋₃alkoxy; X¹ is MgCl, MgBr, MgI, Li, CuLi, ZnX², In(I), or In(X²)₂; each X² independently is Cl, Br, or I; and each X³ independently is Cl, Br, I, OTf, OTs, OAc, or acac.

In various embodiments, R¹ is methyl, ethyl, propyl, n-butyl, or tert-butyl. In some cases, R¹ is methyl, ethyl, or tert-butyl.

In various embodiments, R² is H. In various embodiments, R² is C₁₋₃alkoxy. In some cases, R² is methoxy.

In various embodiments, X¹ is MgCl. In various embodiments, X¹ is MgBr or MgI. In various embodiments, X¹ is Li. In various embodiments, X¹ is CuLi. In various embodiments, X¹ is In(I) or In(X²)₂. In various embodiments, X¹ is ZnCl or ZnBr.

In various embodiments, Zn(X³)₂ is ZnCl₂. In various embodiments, Zn(X³)₂ is ZnBr₂. In various embodiments, Zn(X³)₂ is ZnI₂. In various embodiments, Zn(X³)₂ is Zn(OTf)₂ or Zn(OTs)2. In various embodiments, Zn(X³)₂ is Zn(OAc)₂ or Zn(acac)₂.

In various embodiments, the organic solvent is degassed prior to the admixing. In various embodiments, the organic solvent comprises an ether solvent or acetonitrile. In some cases, the organic solvent is selected from the group consisting of tetrahydrofuran (THF), 2-methyltetrahydrofuran (2-MeTHF), diethyl ether, acetonitrile, 1,2-dimethoxyethane (1,2-DME), methyl tert-butyl ether (MTBE), cyclopentyl methyl ether (CPME), and a combination thereof. In some cases, the organic solvent is acetonitrile.

In various embodiments, the admixing is performed at a temperature of 10° C. to 35° C.

In various embodiments, the admixing comprises (a) admixing compound C and Zn(X³)₂ in the organic solvent to form a suspension; (b) adding

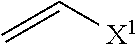

to the suspension to form a solution; and (c) adding compound D to the solution to form compound E. In some cases, the suspension of step (a) is cooled to a temperature of −15° C. to −5° C. prior to adding

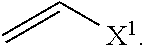

In some cases,

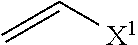

is added to the suspension as a solution in an ether solvent. In some cases, the ether solvent is THF. In some cases,

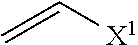

is added to the suspension at a temperature of −10° C. to 0° C. In some cases, the solution of step (b) is brought to a temperature of 10° C. to 35° C. prior to adding compound D. In some cases, compound D is added as a solution in an organic solvent selected from the group consisting of THF, 2-MeTHF, diethyl ether, acetonitrile, 1,2-DME, MTBE, CPME, and a combination thereof. In some cases the organic solvent comprises acetonitrile.

In various embodiments, compound D and

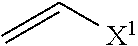

are present in a molar ratio of 1:2.5 to 1:4.5. In some cases, the molar ratio of compound D to

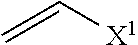

is 1:3.2.

In various embodiments, compound D and Zn(X³)₂ are present in a molar ratio of 1:2.5 to 1:4.0. In various cases, the molar ratio of compound D to Zn(X³)₂ is 1:3.1.

In various embodiments, compound D and compound C are present in a molar ratio of 1:1 to 1:2. In some cases, the molar ratio of compound D to compound C is 1:1.4.

In various embodiments, compound D is prepared by oxidizing compound B:

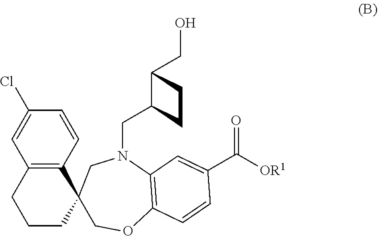

in the presence of an oxidizing agent and an organic solvent. In some cases, the oxidizing occurs under an inert atmosphere.

In various embodiments, compound B is provided as a solution in an organic solvent selected from the group consisting of dimethyl sulfoxide (DMSO), dichloromethane (DCM), dimethylformamide (DMF), THF, 2-MeTHF, acetonitrile toluene, 1,2-DME, MTBE, 1,2-dichloroethane (DCE), chloroform, and a combination thereof. In some cases, the organic solvent is DCM.

In various embodiments, the oxidizing agent is selected from the group consisting of oxalyl chloride, bleach, SO₃/pyridine, iodobenzenediacetate, trifluoroacetic anhydride, N-chlorosuccinimide (NCS), 2-iodooxybenzoic acid (IBX), N-methylmorpholine N-oxide (NMO), ceric ammonium nitrate (CAN), Dess-Martin periodinane, pyridinium chlorochromate (PCC), pyridinium dichromate (PDC), tetrapropylammonium perruthenate (TPAP)/NMO, NCS/dimethylsulfide, NCS/dodecyl sulfide, and a combination thereof. In some cases, the oxidizing agent is oxalyl chloride.

In various embodiments, the oxidizing is performed in the presence of a base selected from the group consisting of triethylamine, diisopropylethanolamine, N-methylpyrrolidine, N-ethylpiperidine, pyridine, 2,2,6,6-tetramethylpiperidine (TMP), pempidine, 2,6-lutidine, and a combination thereof. In some cases, the base is triethylamine.

In various embodiments, compound B and the oxidizing agent are present in a molar ratio of 1:1 to 1:3. In some cases, the molar ratio of compound B to the oxidizing agent is 1:1.5.

In various embodiments, compound B and the base are present in a molar ratio of 1:3 to 1:10. In some cases, the molar ratio of compound B to the base is 1:5.

In various embodiments, the oxidizing occurs in an organic solvent selected from the group consisting of dimethyl sulfoxide (DMSO), dichloromethane (DCM), dimethylformamide (DMF), THF, 2-MeTHF, acetonitrile, MTBE, 1,2-DME, toluene, DCE, CPME, and a combination thereof. In some cases, the organic solvent is DMSO.

In various embodiments, the oxidizing occurs at a temperature of −80° C. to −20° C. In some cases, the oxidizing occurs at a temperature of −40° C.

In various embodiments, the processes further comprise hydrolyzing compound E to form compound F:

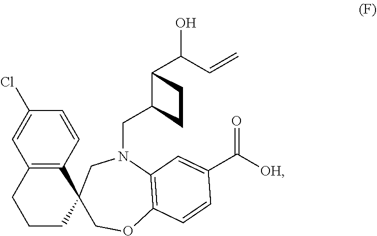

or a salt thereof.

In various embodiments, the hydrolyzing comprises admixing a solution of compound E in an organic solvent and a hydroxide base in water to form compound F.

In various embodiments, the hydroxide base is selected from the group consisting of NaOH, KOH, LiOH, potassium trimethylsilanolate (TMSOK), and a combination thereof.

In various embodiments, compound E and the hydroxide base are present in a molar ratio of 1:1 to 1:100. In some cases, the molar ratio of compound E to the hydroxide base is 1:3.

In various embodiments, the organic solvent is selected from the group consisting of methanol, ethanol, propanol, isopropanol, butanol, THF, diethyl ether, acetone, acetonitrile, 2-MeTHF, sec-butanol, and a combination thereof. In some cases, the organic solvent is ethanol.

In various embodiments, the hydrolyzing occurs at a temperature of 20° C. to 60° F.

In various embodiments, compound F is in salt form. In some cases, the salt of compound F comprises an ammonium cation or an alkali metal cation. In some cases, the ammonium cation is selected from the group consisting of benzylammonium, methylbenzylammonium, trimethylammonium, triethylammonium, morpholinium, pyridinium, piperidinium, picolinium, dicyclohexylammonium, protonated N,N′-dibenzylethylenediamine, 2-hydroxyethylammonium, bis-(2-hydroxyethyl)ammonium, tri-(2-hydroxyethyl)ammonium, protonated procaine, dibenzylpiperidium, dehydroabietylammonium, N,N′-bisdehydroabietylammonium, protonated glucamine, protonated N-methylglucamine, protonated collidine, protonated quinine, protonated quinoline, protonated lysine, protonated arginine, protonated 1,4-diazabicyclo[2.2.2]octane (DABCO), N,N-diisopropylethylammonium, and a combination thereof. In some cases, the ammonium cation is

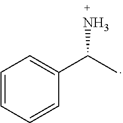

In some cases, the alkali metal cation is selected from the group consisting of lithium, sodium, potassium, and a combination thereof.

In various embodiments, the salt of compound F is prepared by admixing compound F, as its free acid form (compound F free acid), with an amine base or an alkali metal base in a nonpolar organic solvent to form the salt of compound F.

In various embodiments, compound F free acid and amine base or alkali metal base are present in a molar ratio of 1:1 to 1:2. In some cases, the molar ratio of compound F free acid to amine base or alkali metal base is 1:1.2.

In various embodiments, the nonpolar organic solvent is selected from the group consisting of ethyl acetate, toluene, isopropyl acetate, MTBE, and a combination thereof. In some cases, the nonpolar organic solvent is ethyl acetate.

In various embodiments, the admixing (of compound F free acid and the amine base or alkali metal base) occurs at a temperature of 50° C. to 60° C. In some cases, the admixing occurs in an inert atmosphere.

In various embodiments, the processes further comprise synthesizing compound A1 or a salt or solvate thereof using compound E:

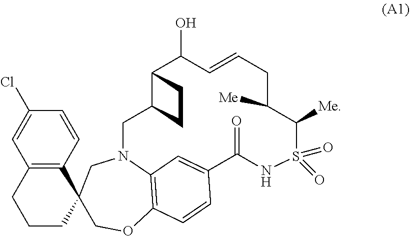

In various embodiments, the processes further comprise synthesizing compound A2 or a salt or solvate thereof using compound E:

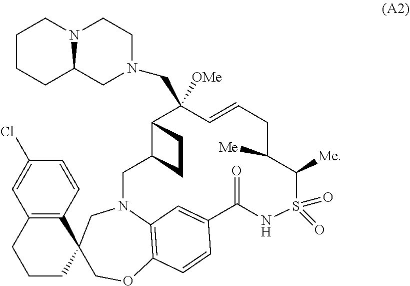

Further aspects and advantages will be apparent to those of ordinary skill in the art from a review of the following detailed description. The description hereafter includes specific embodiments with the understanding that the disclosure is illustrative, and is not intended to limit the invention to the specific embodiments described herein.

### DETAILED DESCRIPTION

Provided herein are processes for synthesizing Mcl-1 inhibitors and corresponding vinylic alcohol intermediates. In particular, processes for synthesizing (1S,3′R,6′R,7′S,8′E,11′S,12′R)-6-chloro-7′-methoxy-11′,12′-dimethyl-3,4-dihydro-2H,15′H-spiro[naphthalene-1,22′[20]oxa[13]thia[1,14]diazatetracyclo[14.7.2.0^{3,6}.0^{19,24}]pentacosa[8,16,18,24]tetraen]-15′-one 13′,13′-dioxide (compound A1), or a salt or solvate thereof, and for synthesizing (1S,3′R,6′R,7′R,8′E,11′S,12′R)-6-chloro-7′-methoxy-11′,12′-dinethyl-7′-((9aR)-octahydro-2H-pyrido[1,2-a]pyrazin-2-ylmethyl)-3,4-dihydro-2H,15′H-spiro[naphthalene-1,22′-[20]oxa[13]thia[1,14]diazatetracyclo [14.7.2.0^{3,6}.0^{19,24}]pentacosa[8,16,18,24]tetraen]-15′-one 13′,13′-dioxide (compound A2), or a salt or solvate thereof are provided:

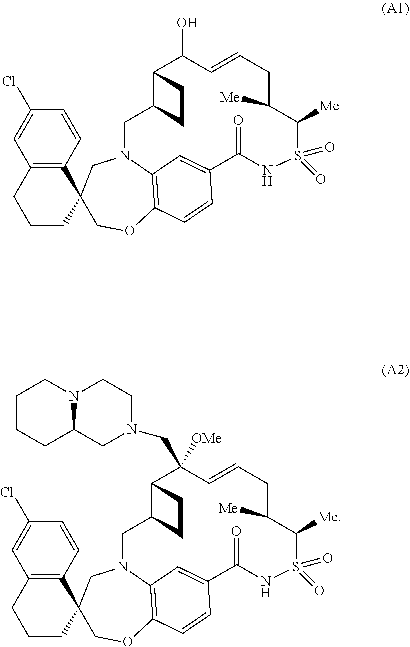

U.S. Pat. No. 9,562,061, which is incorporated herein by reference in its entirety, discloses compound A1, or a salt or solvate thereof, as an Mcl-1 inhibitor and provides a process for preparing it. This patent also discloses a process of synthesizing a vinylic alcohol intermediate compound shown below used in the synthesis of compound A1.

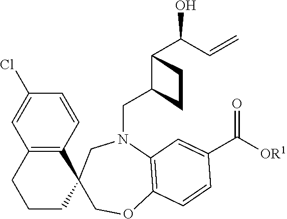

vinylic alcohol intermediate of '061 patent

U.S. Pat. No. 10,300,075, which is incorporated herein by reference in its entirety, discloses compound A2, or a salt or solvate thereof, as an Mcl-1 inhibitor and provides a process for preparing it. The disclosure of compound A2 salts and solvates from U.S. Pat. No. 10,300,075 is incorporated by reference in its entirety. This patent also discloses a process of synthesizing a vinylic alcohol intermediate compound shown above used in the synthesis of compound A2.

The '061 patent generally describes a procedure for making a vinylic alcohol intermediate as shown in Scheme 1, below, which is adapted from the disclosure at col. 49 of the '061 patent. The '061 patent describes that the cyclobutane carbaldehyde (intermediate II) is combined with the oxazepine (intermediate I) in a solvent at a temperature below room temperature preferably 0° C. Sodium cyanoborohydride is added, and the mixture is added to a sodium hydroxide solution, thereby providing intermediate III. Advantageously, the processes described herein provide an improved synthetic route as compared to General Procedure 1 of the '061 patent, as it can be carried out under ambient conditions (e.g., room temperature) and uses milder reagents.

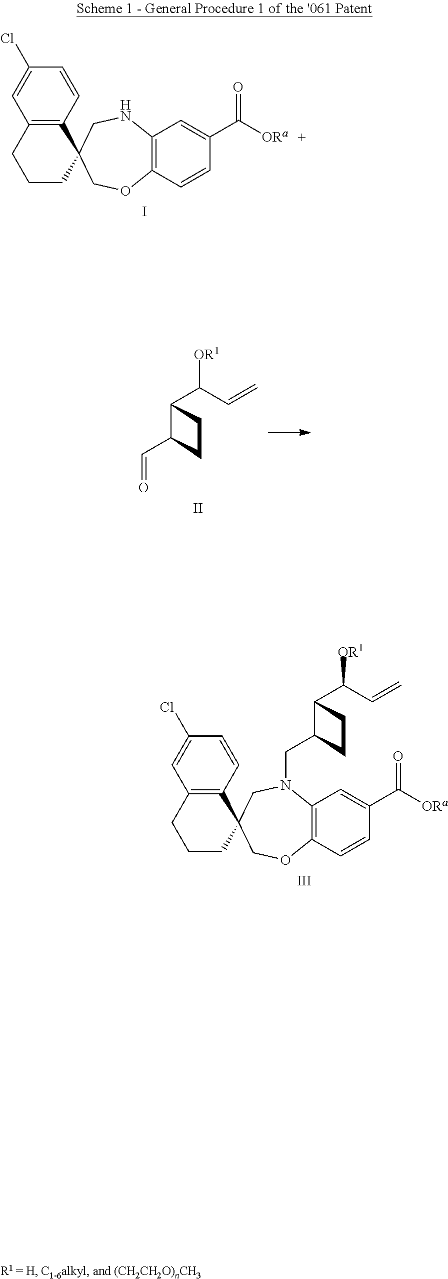

The '061 patent further describes a process for synthesizing the vinylic alcohol intermediate which includes the use of a divinyl zinc reagent in the conversion of the aldehyde intermediate to the vinylic alcohol intermediate. Scheme 2, below, represents the general process of synthesizing the vinylic alcohol as described in the '061 patent.

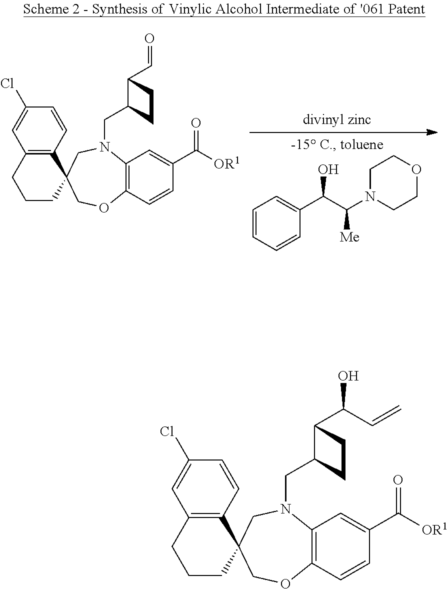

The process of the '061 patent has several disadvantages. Significantly, the divinyl zinc reagent is not commercially available, and therefore must by synthesized prior to use in the reaction. The preparation of the divinyl zinc requires a filtration step to remove inorganic salts, which is not scalable due to the fines clogging. Additionally, the ligand,

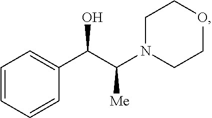 [SMILES: C[C@@H]([C@H](O)c1ccccc1)N1CCOCC1]

must also be synthesized prior to use in the reaction. Moreover, the reaction requires unfavorable cryogenic temperatures and is air- and water-sensitive.

Advantageously, the processes described herein utilize more favorable reaction conditions (i.e., can be performed at or near room temperature) and reagents are more commercially available. For example, cinchonidine and vinyl Grignard reagents are available from natural and/or commercial sources. Moreover, the processes can be carried out in a single reaction vessel without isolation of the intermediates between steps. Higher scalable yields of the final product can also be obtained as compared the process of the '061 patent, as the challenges associated with preparing and storing the divinyl zinc and ligand, as well as the unfavorable reaction conditions, are eliminated.

Described herein are processes for synthesizing compound E or a salt or solvate thereof:

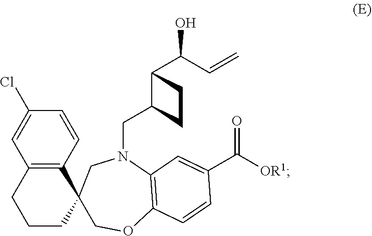

comprising admixing compound C, compound D, and

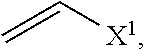

Zn(X³)₂ in an organic solvent to form compound E:

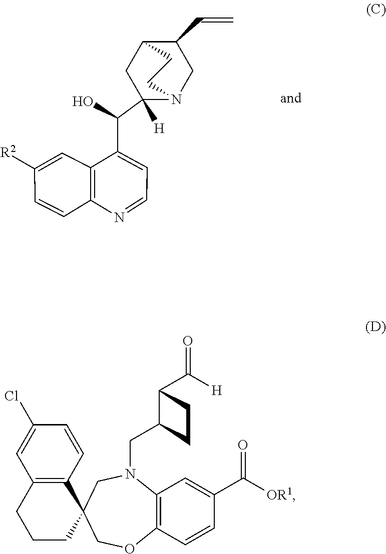

as discussed in detail below. As will be appreciated, the disclosed processes involve formation of a vinylic alcohol intermediate by the addition of a vinyl group across the carbonyl of the corresponding aldehyde intermediate. The processes disclosed herein to form intermediate compounds (e.g., compounds D, E, and F, described in more detail below) can be performed in sequence in a single reaction vessel, without need to isolate the intermediates between steps.

A general reaction scheme for the processes described herein is provided in Scheme 3, below:

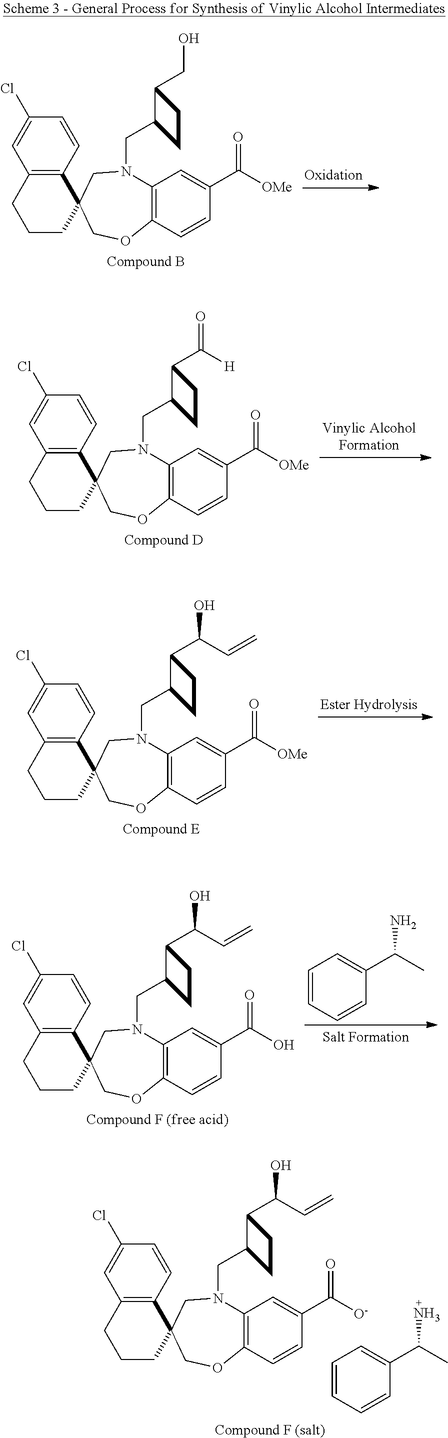

Oxidation

The processes of the disclosure can include oxidizing compound B to provide compound D. In particular, the primary alcohol of compound B can be oxidized to form the aldehyde of compound D. In some embodiments, the oxidizing occurs under an inert atmosphere, for example, under nitrogen or argon gas. In some embodiments, the oxidizing occurs under nitrogen gas.

As provided herein, compound B has a structure of

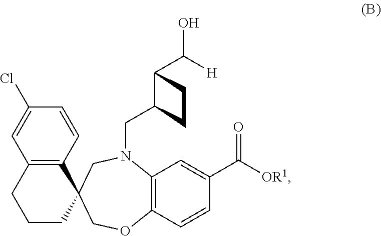

and compound D has a structure of

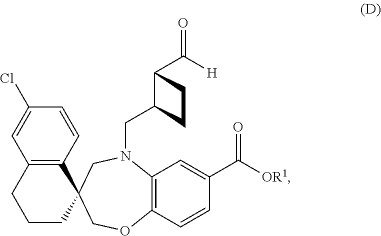

wherein R¹ is C₁₋₆alkyl. As used herein, the term “alkyl” refers to straight chained and branched saturated hydrocarbon groups. The term Cₙ means the group has “n” carbon atoms. For example, C₃ alkyl refers to an alkyl group that has 3 carbon atoms. C₁₋₆ alkyl refers to an alkyl group having a number of carbon atoms encompassing the entire range (i.e., 1 to 6 carbon atoms), as well as all subgroups (e.g., 2-6, 1-5, 1-4, 3-6, 3-5, 1, 2, 3, 4, 5, and 6 carbon atoms). Nonlimiting examples of alkyl groups include methyl, ethyl, n-propyl, isopropyl, n-butyl, sec-butyl (2-methylpropyl), tert-butyl (1,1-dimethylethyl), n-pentyl, and n-hexyl. In some embodiments, R¹ is methyl, ethyl, n-propyl, or tert-butyl. In some embodiments, R¹ is methyl, ethyl, or tert-butyl. In some embodiments, R¹ is methyl. In some embodiments, R¹ is ethyl. In some embodiments, R¹ is tert-butyl.

In some embodiments, compound B is provided as a solution in an organic solvent, e.g., when added to the reaction vessel for the oxidation reaction. Organic solvents are generally known in the art. Nonlimiting examples of organic solvents that can be used throughout the processes described herein include acetonitrile, toluene, benzene, xylene, chlorobenzene, fluorobenzene, naphthalene, benzotrifluoride, tetrahydrofuran (THF), tetrahydropyran, dimethylformamide (DMF), tetrahydrofurfuryl alcohol, diethyl ether, dibutyl ether, diisopropyl ether, methyl tert-butyl ether (MTBE), 2-methyltetrahydrofuran (2-MeTHF), dimethyl sulfoxide (DMSO), 1,2-dimethoxyethane (1,2-DME), 1,2-dichloroethane (1,2-DCE), 1,4-dixoane, cyclopentylmethyl ether (CPME), chloroform, carbon tetrachloride, dichloromethane (DCM), methanol, ethanol, propanol, and 2-propanol.

In some embodiments, compound B is provided as a solution in an organic solvent selected from the group consisting of dimethyl sulfoxide (DMSO), dichloromethane (DCM), dimethylformamide (DMF), THF, 2-MeTHF, acetonitrile, toluene, 1,2-DME, MTBE, 1,2-dichloroethane (1,2-DCE), chloroform, and a combination thereof. In some embodiments, the organic solvent is DCM. That is, in some embodiments, compound B is provided as a solution in DCM.

The oxidation of compound B is performed with an oxidizing agent. Oxidizing agents capable of oxidizing a primary alcohol to an aldehyde are generally known in the art. Nonlimiting oxidizing agents include, but are not limited to, chromium-based reagents, such as Collins reagent (CrO₃.Py₂), pyridinium chlorochromate (PCC), pyridinium dichromate (PDC); sulfonium species (“activated DMSO” resulting from the reaction of DMSO with electrophiles such as oxalyl chloride, a carbodiimide, or SO₃.Py); hypervalent iodine compounds, such as Dess-Martin periodinane (DMP) or 2-iodoxybenzoic acid (IBX); catalytic tetrapropylammonium perruthenate (TPAP) in presence of N-methylmorpholine N-oxide (NMO); and catalytic 2,2,6,6-tetramethylpiperidin-1-yl)oxyl (TEMPO) in presence of NaOCl (bleach).

In some embodiments, the oxidizing agent is selected from the group consisting of oxalyl chloride, bleach, SO₃/pyridine, iodobenzenediacetate, trifluoroacetic anhydride, N-chlorosuccinimide (NCS), 2-iodooxybenzoic acid (IBX), N-methylmorpholine N-oxide (NMO), ceric ammonium nitrate (CAN), Dess-Martin periodinane (DMP), pyridinium chlorochromate (PCC), pyridinium dichromate (PDC), tetrapropylammonium perruthenate (TPAP)/NMO, NCS/dimethylsulfide, NCS/dodecyl sulfide, and a combination thereof. In some embodiments, the oxidizing agent is oxalyl chloride.

Compound B and the oxidizing agent can be present in a molar ratio of 1:1 to 1:3, for example, at least a molar ratio of 1:1, 1:1.25, 1:1.5, 1:1.75, 1:2, or 1:2.25 and/or up to 1:3, 1:2.75, 1:2.5, 1:2.25, 1:2, or 1:1.75, such as 1:1 to 1:2.5, 1:1 to 1:2, 1:1 to 1:1.5, 1:1.25 to 1:2, or 1:1.25 to 1:1.75. In some embodiments, the molar ratio of compound B to the oxidizing agent is 1:1.5.

The oxidation of compound B occurs in the presence of an organic solvent. The organic solvent can be the same or different as the organic solvent used in the solution with compound B. In some embodiments, the oxidation occurs in the presence of an organic solvent selected from the group consisting of dimethyl sulfoxide (DMSO), dichloromethane (DCM), dimethylformamide (DMF), THF, 2-MeTHF, acetonitrile, MTBE, 1,2-DME, toluene, 1,2-DCE, CPME, and a combination thereof. In some embodiments, the oxidation occurs in the presence of DMSO. In some embodiments, the oxidation occurs in the presence of DMSO and DCM.

The organic solvent can be present in an amount of 5 L/kg of compound B to 50 L/kg of compound B, for example, at least 5, 10, 15, 20, 25, or 30 L/kg of compound B and/or up to 50, 45, 40, 35, 30, 25, or 20 L/kg of compound B, such as 10 to 40 L/kg of compound B, 15 to 30 L/kg of compound B, or 15 L/kg to 20 L/kg of compound B.

The oxidation of compound B can be performed in the presence of a base, for example, an amine base (e.g., mono-, di-, or trialkylamines, substituted or unsubstituted piperidines, substituted or unsubstituted pyridines, etc.). In some embodiments, the base is selected from the group consisting of triethylamine, diisopropylethanolamine, N-methylpyrrolidine, N-ethylpiperidine, pyridine, 2,2,6,6-tetramethylpiperidine (TMP), pempidine, 2,6-lutidine, and a combination thereof. In some embodiments, the base is triethylamine.

When a base is present in the oxidation of compound B, compound B and the base can be present in a molar ratio of 1:3 to 1:10, for example, at least 1:3, 1:4, 1:5, 1:6, or 1:7, and/or up to 1:10, 1:9, 1:8, 1:7, or 1:6, such as 1:3 to 1:9, 1:5 to 1:10, 1:4 to 1:8, or 1:4 to 1:6. In some embodiments, the molar ratio of compound B to the base is 1:5.

The oxidation of compound B can occur at a temperature of −80° C. to −20° C., for example at least −80, −70, −60, −55, −50, −45, or −40° C. and/or up to −20, −25, −30, −35, −40, −50, or −60° C., such as −70° C. to −25° C., −60° C. to −30° C., −50° C. to −30° C., or −45° C. to −35° C. In some embodiments, the oxidizing occurs at a temperature of −40° C.

In some embodiments, compound B and/or compound D is a salt. A salt of compound B, compound D, or any other compound described herein can be prepared, for example, by reacting the compound in its free acid form (e.g., when R¹ is H) with a suitable organic or inorganic base, and optionally isolating the salt thus formed. Nonlimiting examples of suitable salts include alkali metal cation, such as lithium, sodium, potassium, and combinations thereof, or an ammonium cation, such as benzylammonium, methylbenzylammonium, trimethylammonium, triethylammonium, morpholinium, pyridinium, piperidinium, picolinium, dicyclohexylammonium, protonated N,N′-dibenzylethylenediamine, 2-hydroxyethylammonium, bis-(2-hydroxyethyl)ammonium, tri-(2-hydroxyethyl)ammonium, protonated procaine, dibenzylpiperidium, dehydroabietylammonium, N,N′-bisdehydroabietylammonium, protonated glucamine, protonated N-methylglucamine, protonated collidine, protonated quinine, protonated quinoline, protonated lysine, protonated arginine, protonated 1,4-diazabicyclo[2.2.2]octane (DABCO), N,N-diisopropylethylammonium, amino acid salts, and the like. In some embodiments, compound B, compound D, or any other compound described herein can be prepared, for example, by reacting the compound in its free form with a suitable organic or inorganic acid, and optionally isolating the salt thus formed. Nonlimiting examples of suitable acid salts include hydrobromide, hydrochloride, sulfate, bisulfate, sulfonate, camphorsulfonate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like.

The oxidation of compound B provides compound D which can be through-processed directly into the next step without the need for separation.

Vinylic Alcohol Formation

The processes of the disclosure include admixing compound C, compound D (e.g., as prepared in Step 1),

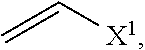

and Zn(X³)₂ in an organic solvent to form compound E:

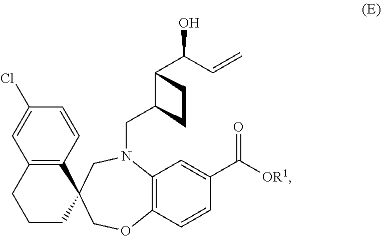

wherein R¹ is as described above, X¹ is MgCl, MgBr, MgI, Li, CuLi, ZnX², In(I), or In(X²)₂; each X² independently is Cl, Br, or I; and, each X³ independently is Cl, Br, I, triflate (OTf), tosylate (OTs), acetate (OAc), or 2,4-acetylacetonate (acac).

Advantageously, the processes of the disclosure can use commercially available reagents in the synthesis of the vinylic alcohol intermediates (e.g. compound E) from the corresponding aldehyde (e.g., compound D), thereby precluding an additional and separate synthesis of, for example, the divinyl zinc used in the process of U.S. Pat. No. 9,562,061.

As provided herein, compound C has a structure of

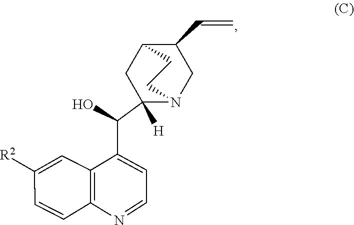

wherein R² is H or C₁₋₃alkoxy. In some embodiments, R² is H (i.e., compound C is cinchonidine). In some embodiments, R² is C₁₋₃ alkoxy. As used herein, the term “alkoxy” is defined as —OR, wherein R is an alkyl group. For example, R² can be methoxy (—OCH₃), ethoxy (—OCH₂CH₃), n-propoxy (—OCH₂CH₂CH₃), or isopropoxy (—OCH(CH₃)₂). In some embodiments, R² is methoxy compound C is quinine).

The vinylic reagent,

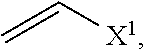

can be any one or a Grignard reagent, an organolithium reagent, an organocuprate reagent, an organozinc reagent, or an organoindium reagent that is suitable for addition of the vinylic group across the aldehyde of compound D.

In some embodiments,

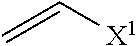

is a Grignard reagent. A “Grignard reagent” means that X¹ includes a magnesium with a halogen, such as Cl, Br, or I. In some embodiments, X¹ is MgCl. In some embodiments, X¹ is MgBr or MgI.

In some embodiments,

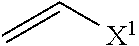

is an organolithium reagent. For example, in some embodiments, X¹ is Li. In some embodiments,

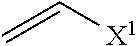

is an organocuprate reagent. For example, in some embodiments, X¹ is CuLi. In some embodiments,

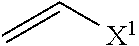

is an organoindium reagent. For example, in some embodiments, X¹ is In(I) or In(X²)₂. In some embodiments, X¹ is In(I). In some embodiments, X¹ is In(X²)₂, wherein each X² independently is Cl, Br, or I. In some embodiments, X¹ is InCl₂. In some embodiments, X¹ is InBr₂. In some embodiments, X¹ is InI₂. In some embodiments,

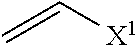

is an organozinc reagent. For example, in some embodiments, X¹ is ZnX², wherein X² is as described herein. In some embodiments, X¹ is ZnCl or ZnBr. In some embodiments, X¹ is ZnCl. In some embodiments, X¹ is ZnBr.

Compound D and

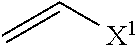

can be present in a molar ratio of 1:2.5 to 1:4.5, for example at least 1:2.5, 1:2.75, 1:3, 1:3.25, 1:3.5, or 1:3.75 and/or up to 1:4.5, 1:4.0, 1:3.75, 1:3.5, 1:3.25, or 1:3, such as 1:2.5 to 1:4, 1:3 to 1:4.5, 1:3 to 1:4, or 1:3 to 1:3.5. In some embodiments, the molar ratio of compound D to

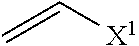

is 1:3.2.

As provided herein the Processes include admixing Zn(X³)₂ with compound C, compound D, and

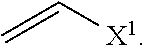

In some embodiments, Zn(X³)₂ is ZnCl₂. In some embodiments, Zn(X³)₂ is ZnBr₂. In some embodiments, Zn(X³)₂ is ZnI₂. In some embodiments, Zn(X³)₂ is Zn(OTf)₂. In some embodiments, Zn(X³)₂ is Zn(OTs)₂. In some embodiments, Zn(X³)₂ is Zn(OAc)₂. In some embodiments, Zn(X³)₂ is Zn(acac)₂.

Compound D and Zn(X³)₂ can be present in a molar ratio of 1:2.5 to 1:4, for example at least 1:2.5, 1:2.75, 1:3, or 1:3.25 and/or up to 1:4, 1:3.75, 1:3.5, 1:3.25 or 1:3, such as 1:2.5 to 1:3.5, 1:2.75 to 1:3.5, 1:3 to 1:4, or 1:3 to 1:3.5. In some embodiments, the molar ratio of compound D to Zn(X³)₂ is 1:3.1.

The admixing of compounds C, compound D,

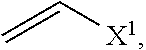

and Zn(X³)₂ occurs in an organic solvent. In some embodiments, the organic solvent is an ether solvent or acetonitrile. Nonlimiting examples of ether solvents include, for example, tetrahydrofuran (THF), 2-methyltetrahydrofuran (2-MeTHF), tetrahydropyran, tetrahydrofurfuryl alcohol, diethyl ether, dibutyl ether, diisopropyl ether, methyl tert-butyl ether (MTBE), 1,2-dimethoxyethane, 1,4-dixoane, 2-methyl-THF, and cyclopentylmethyl ether. In some embodiments, the organic solvent is selected from the group consisting of tetrahydrofuran (THF), 2-methyltetrahydrofuran (2-MeTHF), diethyl ether, 1,2-dimethoxyethane (1,2-DME), methyl tert-butyl ether (MTBE), cyclopentylmethylether (CPME), and a combination thereof. In some embodiments, the organic solvent is acetonitrile.

The admixing can occur at a temperature of 10° C. to 35° C., for example at least 10, 15, 20, or 25° C. and/or up to 35, 30, 25, or 20° C., for example 15° C. to 30° C., or 20° C. to 25° C.

In some embodiments, the admixing includes (a) admixing compound C and Zn(X³)₂ in the organic solvent to form a suspension, (b) adding

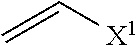

to the suspension to form a solution, and (c) adding compound D to the solution to form compound E.

In some embodiments, the suspension of step (a) is cooled to a temperature of −15° C. to −5° C. prior to adding

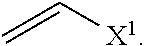

For example, the suspension of step (a) can be cooled to a temperature of −12° C. to −7° C., or −10° C. to −8° C. In some embodiments, the suspension of step (a) is cooled to a temperature of −10° C. before adding

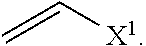

In some embodiments,

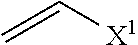

is added to the suspension as a solution in an ether solvent, for example, in tetrahydrofuran (THF), 2-methyltetrahydrofuran (2-MeTHF), tetrahydropyran, tetrahydrofurfuryl alcohol, diethyl ether, dibutyl ether, diisopropyl ether, methyl tert-butyl ether (MTBE), 1,2-dimethoxyethane, 1,4-dixoane, 2-methyl-THF, or cyclopentylmethyl ether. In some embodiments,

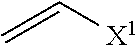

is added to the suspension as a solution in THF.

In some embodiments,

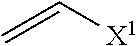

is added to the suspension at a temperature of −10° C. to 0° C., for example at least −10, −9, −8, −7, −6, −5, or −4 and/or up 0, −1, −2, −3, −4, −5, or −6° C., such as −8° C. to 0° C., −6° C. to −2° C., or −6 to −° C. In some embodiments,

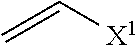

is added to the suspension at a temperature of −5° C. The solution of step (b) can be brought to a temperature of 1 ° C. to 35° C. prior to adding compound D (e.g., after adding

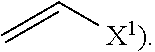

For example, the solution of step (b) can be brought to a temperature of 10, 15, 20, 25, or 30° C. and/or up to 35, 30, 25, 20 or 15° C., such as 15° C. to 30° C., 15° C. to 25° C., or 20° C. to 25° C. prior to adding compound D. In some embodiments, the solution of step (b) is brought to a temperature of 20° C. prior to adding compound D.

Compound D can be added in step (c) as a solution in an organic solvent. For example, compound D can be added as a solution in an organic solvent selected from the group consisting of THF, 2-MeTHF, diethyl ether, acetonitrile, 1,2-DME, MTBE, CPME, and a combination thereof. In some embodiments, compound D is added as a solution in acetonitrile.

The organic solvent can be present in an amount of 5 L/kg of compound D to 30 L/kg of compound D, for example, at least 5, 7, 10, 12, 15, 17, 20 or 22 L/kg of compound D and/or up to 30, 27, 25, 22, 20, or 15 L/kg of compound D, such as 10 to 30 L/kg of compound D, 15 to 30 L/kg of compound D, or 10 L/kg to 20 L/kg of compound D.

In some embodiments, compound E is a salt. Salts of compound E can be similar to those as described herein for compound B or D.

Compound E:

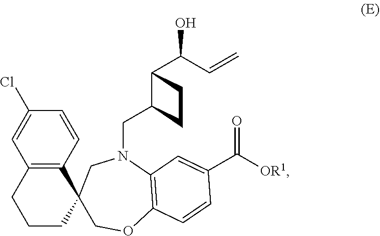

wherein R¹ is as described herein, can be through-processed directly into the next step without the need for separation.

Ester Hydrolysis and Salt Formation

The processes of the disclosure can further include hydrolyzing the ester compound E to form compound F:

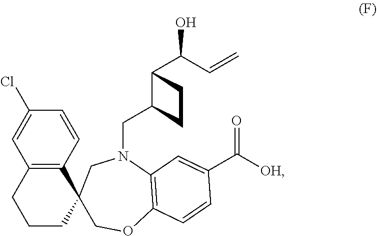

or a salt thereof.

In some embodiments, the hydrolyzing includes using an enzyme (e.g., enzymatic hydrolysis). In some embodiments, the hydrolyzing includes admixing a solution of compound E in an organic solvent and a hydroxide base in water to form compound F. Nonlimiting examples of hydroxide bases include sodium hydroxide, potassium hydroxide, lithium hydroxide, potassium trimethylsilanoate (TMSOK). In some embodiments, the hydroxide base is selected from the group consisting of NaOH, KOH, LiOH, TMSOK, and a combination thereof. In some embodiments, the hydroxide base is NaOH.

Compound E and the hydroxide base can be present in a molar ratio of 1:1 to 1:100, for example at least 1:1, 1:5, 1:10, 1:15, 1:20, 1:25, 1:30, 1:40, 1:50 or 1:60 and/or up to 1:100, 1:95, 1:90, 1:80, 1:75, 1:70, 1:60, 1:50, 1:45, or 1:40, such as 1:1 to 1:75, 1:1 to 1:50, 1:1 to 1:25, 1:1 to 1:10, or 1:1 to 1:5. In some embodiments, the molar ratio of compound E to the hydroxide base is 1:3.

The hydrolysis can be performed in the presence of an organic solvent, for example any organic solvent as described herein, such as an ether solvent, an alcohol solvent (e.g., methanol, ethanol, propanol, butanol, etc.), or any water-miscible solvent (e.g., THF, acetonitrile, etc.). In some embodiments, the organic solvent is selected from the group consisting of methanol, ethanol, propanol, isopropanol, butanol, THF, diethyl ether, acetone, acetonitrile, 2-MeTHF, sec-butanol, and a combination thereof. In some embodiments, the organic solvent is ethanol.

The hydrolyzing can occur at a temperature of 20° C. to 60° F., for example, at least 20, 25, 30, 35, 40, or 45° C. and/or up to 60, 55, 50, 45, 40 or 35° C., such as 25° C. to 60° C., 30° C. to 60° C., 40° C. to 60° C., or 50° C. to 60° C. In some embodiments, the hydrolyzing occurs at a temperature of 55° C.

Once hydrolysis is complete, the solution can be cooled or otherwise brought to ambient room temperature (e.g., 15, 20, or 25° C.), at which point the reaction can be neutralized to a pH of 6-7 with an acid, such as phosphoric acid.

The hydrolysis can provide compound F in its free acid form:

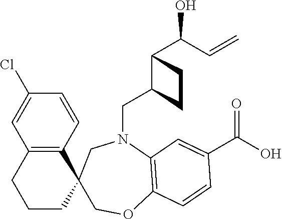

(F free acid).

The processes of the disclosure can further include providing compound F in a salt form. For example, compound F in a salt form can have a structure of:

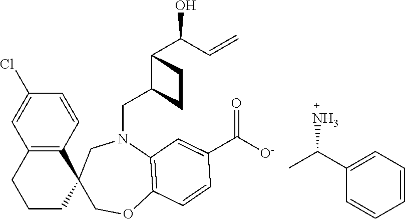

(F salt form).

In some embodiments, the salt of compound F can include an ammonium cation or an alkali metal cation. In some embodiments, the salt of compound F includes an alkali metal cation, such as lithium, sodium, potassium, and combinations thereof. In some embodiments, the salt of compound F includes an ammonium cation, such as benzylammonium, methylbenzylammonium, trimethylammonium, triethylammonium, morpholinium, pyridinium, piperidinium, picolinium, dicyclohexylammonium, protonated N,N′-dibenzylethylenediamine, 2-hydroxyethylammonium, bis-(2-hydroxyethyl)ammonium, tri-(2-hydroxyethyl)ammonium, protonated procaine, dibenzylpiperidium, dehydroabietylammonium, N,N′-bisdehydroabietylammonium, protonated glucamine, protonated N-methylglucamine, protonated collidine, protonated quinine, protonated quinoline, protonated lysine, protonated arginine, protonated 1,4-diazabicyclo[2.2.2]octane (DABCO), N,N-diisopropylethylammonium, and combinations thereof. In some embodiments, the ammonium cation is

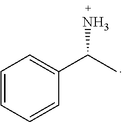

The salt of compound F can be prepared by admixing compound F, as its free acid form (compound F free acid) with an amine base or an alkali metal base in a nonpolar organic solvent to form the salt of compound F (compound F salt form).

Nonlimiting examples of amine bases include alkylamines, such as mono-, di, or trialkylamines (e.g., monoethylamine, diethylamine, triethylamine, and N,N-diisopropylethylamine), pyridines, such as collidine and 4-diethylaminopyridine (DMAP), and imidazoles, such as N-methylimidazole, as well as benzylamine, methylbenzylamine, morpholine, piperidine, picoline, dicyclohexylamine, N,N′-dibenzylethylenediamine, 2-hydroxyethylamine, bis-(2-hydroxyethy)amine, tri-(2-hydroxyethyl)amine, procaine, dibenzylpiperidine, dehydroabietylamine, N,N′-bisdehydroabietylamine, glucamine, N-methylglucamine, quinine, quinoline, lysine, arginine, 1,4-diazabicyclo[2.2.2]octane (DABCO), and N,N-diisopropylethylamine. Nonlimiting examples of alkali metal bases include NaOH, LiOH, and KOH.

Compound F free acid and the amine base or alkali metal base can be present in a molar ratio of 1:1 to 1:2, for example at least 1:1, 1:1.1, 1:1.2, 1:1.3, 1:1.4, 1:1.5, or 1:1.6 and/or up to 1:2, 1:1.9, 1:1.8, 1:1.7, 1:1.6, 1:1.5, or 1:1.4, such as 1:1 to 1:7, 1:1 to 1:5, or 1:1 to 1:1.3. In some embodiments, the molar ratio of compound F free acid to the amine base or alkali metal base is 1:1.2.

Compound F free acid can be admixed with the amine base or alkali metal base in a nonpolar organic solvent. In some embodiments, the nonpolar organic solvent is selected from the group consisting of ethyl acetate, toluene, isopropyl acetate, MTBE, and a combination thereof. In some embodiments, the nonpolar organic solvent is ethyl acetate.

Compound F free acid and the amine base or alkali metal base can be admixed at a temperature of 50° C. to 60° C., for example, at least 50, 52, 55, or 57° C. and/or up to 60, 57, 55, or 52° C., such as 52° C. to 60° C., 55° C. to 60° C., or 57 C to 60° C. In some embodiments, the admixing occurs at a temperature of 60° C.

The admixing can occur in an inert atmosphere, for example, under nitrogen or argon gas. In some embodiments, the admixing is performed under nitrogen gas.

The admixing of compound F free acid with the amine base or alkali metal base in the nonpolar organic solvent provides compound F salt form, which can be crystallized for later use, for example in the synthesis of compound A1 or A2.

The processes for synthesizing compounds E and F can be used to synthesize compounds A1 and A2 from compound E and F. As shown in Scheme 4 below, compounds E and F may be used to synthesize compound A1 and salts and solvates thereof and as shown in Scheme 5, compounds E and F may also be used to synthesize compound A2 and salts and solvates thereof.

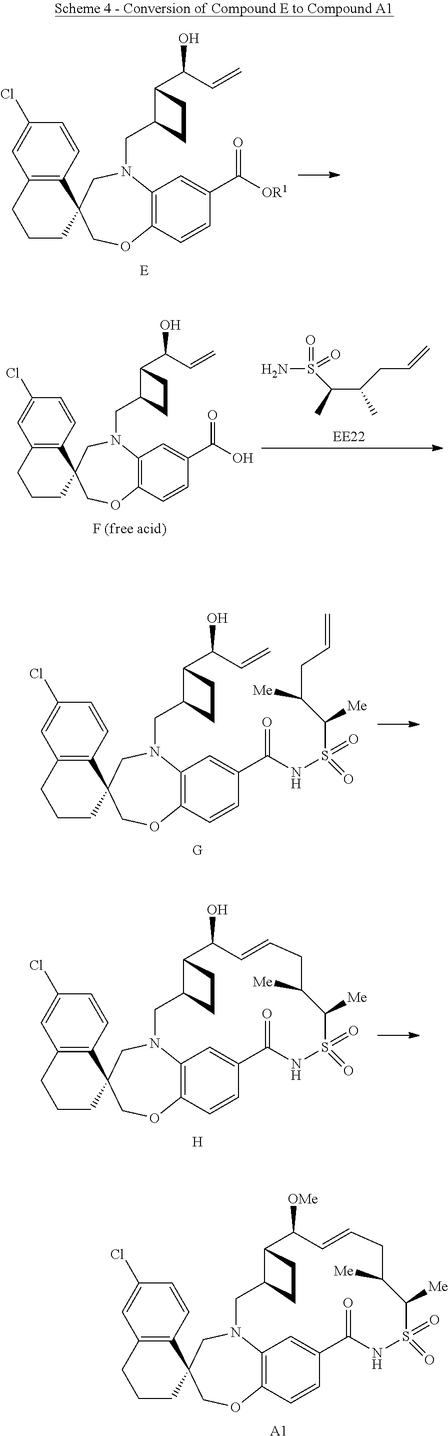

As shown in Scheme 4 and described in U.S. Pat. No. 9,562,061, compounds E and F may be used to synthesize compound A1 and salts and solvates thereof. The synthesis of sulfonamide EE22 is disclosed in U.S. Pat. No. 9,562,061. As described herein, compound E can be used to prepare compound F by conversion of the ester E to the carboxylic acid F. As set forth in U.S. Pat. No. 9,562,061, compounds EE22 and compound F can be reacted to form compound G. Cyclization of compound G can provide hydroxy compound H which can then be methylated to provide compound A1 as described in U.S. Pat. No. 9,562,061.

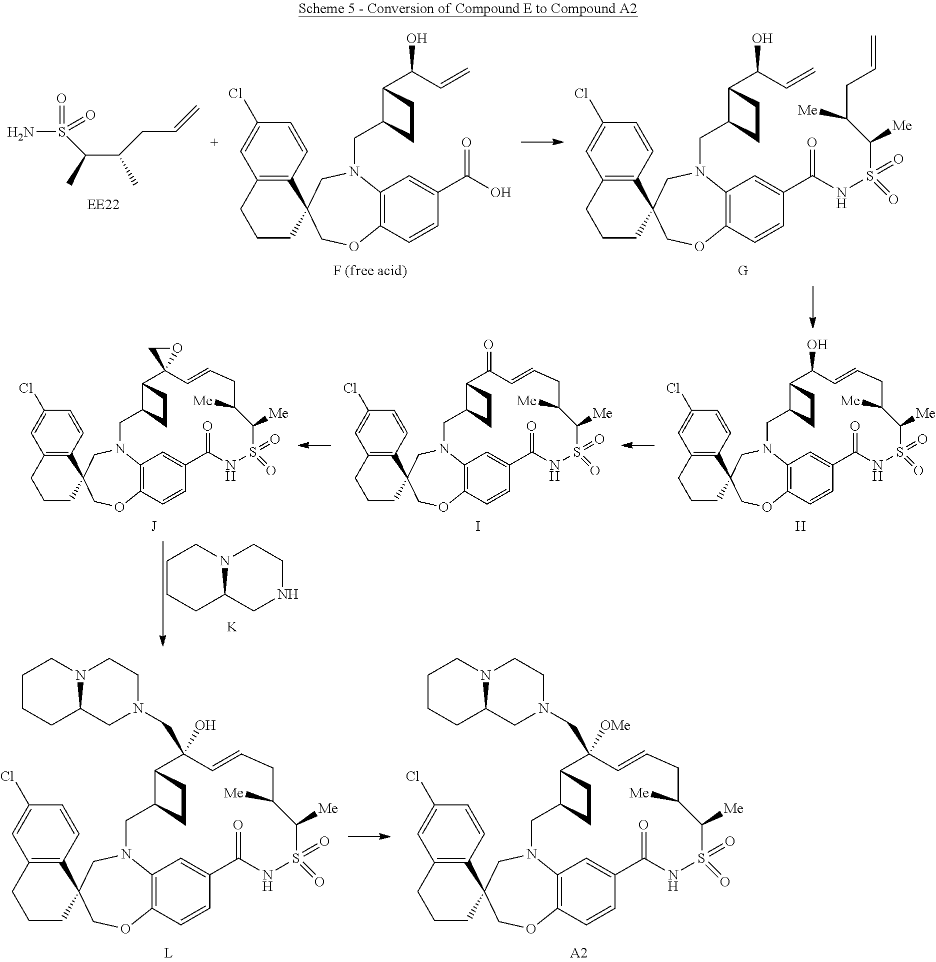

As shown in Scheme 5 and described in U.S. Pat. No. 10,300,075, compounds E and F can be used to synthesize compound A2 and salts and solvates thereof. As described above with respect to Scheme 4, the synthesis of sulfonamide EE22 is disclosed in U.S. Pat. No. 9,562,061. Also as described above and set forth in U.S. Pat. No. 9,562,061, sulfonamide EE22 and compound F can be reacted to form compound G which can be cyclized to produce hydroxy compound H. Compound H can then be oxidized to provide cyclic enone I as disclosed in U.S. Pat. No. 10,300,075. Alternatively, compound G can be oxidized to provide the uncyclized enone version of compound G and then cyclized to provide cyclic enone I. Enone I can then be converted to epoxide J using the procedures disclosed in U.S. Pat. No. 10,300,075. Epoxide J can then be reacted with bicyclic compound K to provide hydroxy compound L. Finally, methylation of compound L can provide compound A2 as disclosed in U.S. Pat. No. 10,300,075.

In some embodiments, the processes further include synthesizing compound A1 or a salt or solvate thereof using compound D:

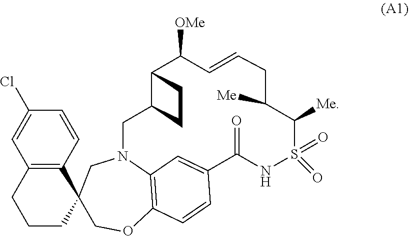

In some embodiments, the processes further include synthesizing compound A2 or a salt or solvate thereof using compound D:

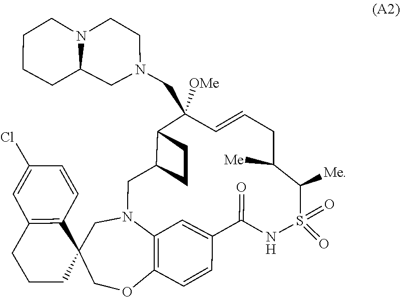

It is to be understood that while the disclosure is read in conjunction with the detailed description thereof, the foregoing description and following example are intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the invention.

### Example 1: Oxidation

Methyl-(S)-6′-chloro-5-(((1R,2R)-2-formylcyclobutyl)methyl)-3′,4,4′,5-tetrahydro-2H,2′H-spiro[benzo[b][1,4]oxazepine−3,1′-naphthalene]-7-carboxylate) was prepared according to the following reaction scheme:

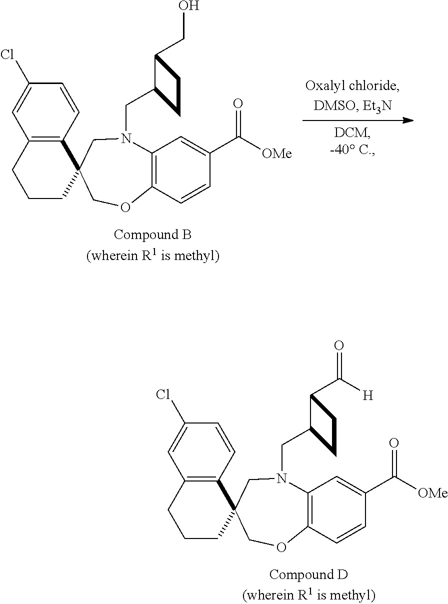

To a 1200 L reactor under nitrogen was charged dichloromethane (125 L, 15 L/kg) and dimethylsulfoxide (DMSO) (4.265 Kg, 3 eq.). The resulting mixture was cooled to −40° C. and oxalyl chloride (3.465 Kg, 1.5 eq.) was added in 1 hour, maintaining the temperature below −35 ° C. The resulting solution was stirred for 30 minutes at −35° C. then a solution of Compound B (8.3 kg, 18.2 mol, 1.0 equiv) in dichloromethane (38 L, 4.6 L/kg) was added in 0.7 hr, maintaining the temperature at −35° C. After 30 minutes stirring, triethylamine (9.20 Kg, 5 eq.) was introduced at −35° C. over a period of 0.7 hr. The suspension was stirred at −35° C. for 0.8 hour then the reaction was monitored by HPLC. Stirring at −35° C. was maintained for 0.6 hours, then additional oxalyl chloride (462 g, 0.2 eq.) was added at −35° C. in 18 min and complete conversion was confirmed. The reaction mixture was allowed to warm to −13° C. and deionized water (41.5 L, 5 L/kg) was added in 16 minutes maintaining the temperature below 0 ° C. The resulting biphasic solution was stirred for 20 min then allowed to settle. Layers were separated and the organic layer was transferred into a 250 L enameled reactor. The solution was washed with 1N HCl (5 L/kg) followed by a sodium bicarbonate solution (5 L/kg) and then a sodium chloride solution (5 L/kg). The organic layer was dried over sodium sulfate (8.3 Kg, 1 eq. w/w %), filtered and the solid was washed with dichloromethane (2×25 L, 2×3 L/kg.). Dichloromethane was removed by atmospheric distillation at 40° C. to a minimum stirring volume and acetonitrile was added (120 L, 15 L/kg). Concentration was continued under vacuum at 40 ° C. in order to remove residual water and dichloromethane. Compound D was obtained as a solution in acetonitrile in quantitative yield and through processed directly into the next step.

### Example 2: Vinylic Alcohol Formation

Methyl (S)-6′-chloro-5-(((1R,2R)-2-(S)-1-hydroxyallyl)cyclobutyl)methyl)-3′,4,4′,5-tetrahydro-2H,2′H-spiro[benzo[b][1,4]oxazepine-3,1′-naphthalene]-7-carboxylate (compound E) was prepared according to the following reaction scheme:

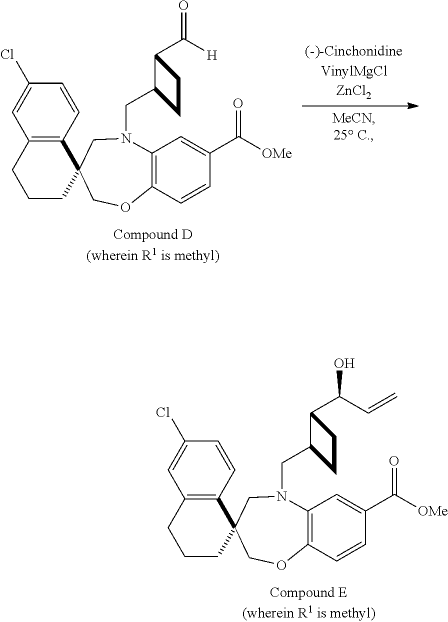

To a 250 L enameled reactor was charged acetonitrile (54 L, 13.1 L/kg.). The solvent was degassed by nitrogen bubbling, then to it was charged cinchonidine (3.75 Kg, 1.4 eq.), and zinc chloride (384 g, 3.1eq.) was added in 1 to 1.5 hr to the suspension, maintaining the temperature below 28° C. The resulting solution was cooled to −10° C. and vinylmagnesium chloride solution in THF (15.10 Kg, 3.2 eq.) was added at −5±5° C. over a period of 0.8 to 1.2 hr. The reaction mixture was warmed to 20° C. in 0.8 hr, then a solution of Compound D in acetonitrile (23.30 Kg, 4.12 Kg pure, 1.0 eq.) was added in 5 min at 20° C. The reaction mixture was stirred for 0.5 hr at that temperature. The reaction was monitored by HPLC. Toluene (26 L, 6.4 L/kg) and 1.5 M citric acid solution were added. The biphasic solution was stirred for 20 min, then the layers were allowed to settle. After separation, the organic layer was washed with additional 1.5 M citric acid solution, then brine. The solution was concentrated at atmospheric pressure to 80 L of residual solution. The solution was cooled to 35° C. then transferred into a cleaned 250 L enameled reactor. Concentration was continued to 20 L of residual volume, and ethanol (85 L) was added. Concentration was continued in order to remove residual acetonitrile and toluene. Compound E was obtained as a solution in ethanol and through processed directly into the next step.

### Example 3: Ester Hydrolysis

(S)-6′-chloro-5-(((1R,2R)-2-((S)-1-hydroxyally)cyclobutyl)methyl)-3′,4,4′,5-tetrahydro-2H,2′H-spiro[benzo[b][1,4]oxazepine-3,1′-naphthalene]-7-carboxylic acid (compound F free acid) was prepared according to the following reaction scheme:

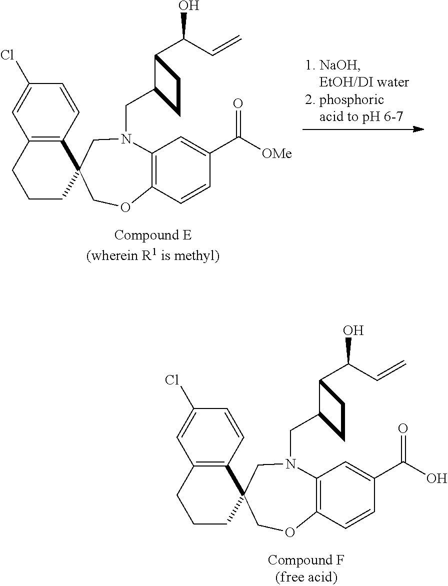

To a 250 L enameled reactor under nitrogen was charged with a solution of Compound E (9 kg) in ethanol. The mixture was heated at 55±5° C. and deionized water (9 L, 1 L/kg) was added. A mixture of 30.5% w/w sodium hydroxide solution (7.1 Kg, 2.9 eq.) and deionized water (9 L, 1 L/kg) was added at 55±5° C. in 15 minutes. The resulting solution was stirred at 55±5° C. for 1.7 hr. After confirming complete conversion by HPLC, the solution was cooled to 20±5° C. and phosphoric acid (74.7 1.9 Kg, 0.8 eq.) was added at 20±5° C. in 15 min until pH is 6-7. Ethyl acetate (41 L, 4.7 L/kg) was added and stirring was continued for 15 min. The biphasic mixture was allowed to settle and layers separated. The organic layer was washed twice with brine, then concentrated at atmospheric pressure to 25 L of residual volume. Ethyl acetate (130 L) was added and azeotropic distillation was continued to 25 L of residual volume. The mixture was filtered through thick paper filter under nitrogen pressure to remove precipitates. The reactor and filter were rinsed with ethyl acetate (2×10 L, 2×1.1 L/kg). Filtrates were combined and stored in a drum under nitrogen. Compound F free acid was obtained through processed directly into the next step.

¹H NMR (400 MHz, DMSO-d6) δ1.36 -2.15 (m, 9H), 2.37 -2.55 (m, 1H) 2.61 -2.83 (m, 2H) 3.16 -3.35 (m, 2H) 3.44 (br s, 2H) 4.00 (br d, J=4.15 Hz, 3H) 4.52 -4.86 (m, 1H) 4.90 -5.03 (m, 1H) 5.09 -5.26 (m, 1H) 5.63 -5.85 (m, 1H) 6.89 (br d, J=8.09 Hz, 1H) 7.02 −7.33 (m, 3H) 7.40 (br s, 1H) 7.62 (br d, J=8.50 Hz, 1H) 12.13 -12.98 (m, 1H). LRMS (ESI): Calculated. for C₂₇H₃₀CINO₄+H: 468.2, Found: 468.2.

### Example 4: Salt Formation

(S)-6′-chloro-5-(((1R,2R)-2-((S)−1-hydroxyallyl)cyclobutyl)methyl)-3′,4,4′,5-tetrahydro-2H,2′H-spiro[benzo[b][1,4]oxazepine-3,1′-naphthalene]-7-carboxylate, (R)-1-phenylethan-1-aminium salt (compound F salt form) was prepared according to the following reaction scheme:

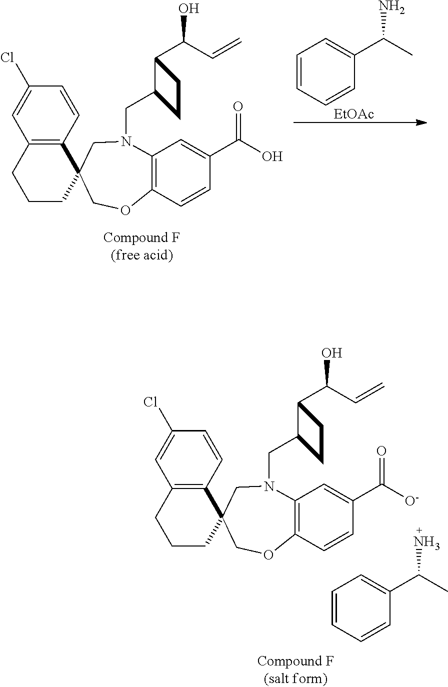

To a 250 L enameled reactor under nitrogen was charged with Compound F (free acid) solution in ethyl acetate (44.1 Kg, 7.88 Kg pure, 1 eq.) and ethyl acetate (39 L, adjusting to 10 L/kg). The resulting solution was heated to 60° C. and (R)-(+)-α-methylbenzylamine (2448 g, 1.2 eq.) was added in 13 minutes at that temperature. When the reaction mixture became slightly turbid (after 4/5 of the amine addition), crystallization was seeded with Compound F salt form. The resulting solution was stirred at 60±5° C. for 1 hour then cooled to 22±3° C. over 45 min. The mixture was held for at least 45 min prior to filtration under vacuum. Reactor and filter cake were washed with ethyl acetate (2×8 L, 2>1 L/kg) and the solid was dried under vacuum at 45° C. overnight. After sieving, Compound F salt form was obtained.

¹H NMR (400 MHz, DMSO-d6) δ7.60 -7.69 (m, 3H), 7.46 -7.53 (m, 3H), 7.32 -7.39 (m, 2H), 7.29 (s, 2H), 7.20 (dd, J=8.50, 2.28 Hz, 1H), 7.15 (d, J=2.28 Hz, 1H), 6.82 (d, J=8.09 Hz, 1H), 5.78 (ddd, J=17.21, 10.47, 5.49 Hz, 1H), 5.14 -5.21 (m, 1H), 4.94 -4.99 (m, 1H), 4.30 (q, J=6.63Hz, 1H), 3.91 -4.06 (m, 3H), 3.57 (br d, J=12.02Hz, 1H), 3.41 (br d, J=14.10 Hz, 1H), 3.14 -3.26 (m, 2H), 2.65 -2.81 (m, 2H), 2.41 -2.50 (m, 1H), 1.88 -2.07 (m, 3H), 1.75 -1.86 (m, 2H), 1.68 -1.77 (m, 1H), 1.50 -1.65 (m, 3H), 1.44 -1.50 (m, 3H); ¹³C NMR (100 MHz, DMSO-d6) δ169.9, 150.9, 142.5, 140.6, 140.4, 139.6, 139.4, 131.6, 130.8, 129.6, 128.4, 128.2, 127.5, 126.5, 126.0, 120.3, 119.4, 117.6, 113.4, 78.8, 75.1, 61.3, 59.0, 50.0, 45.0, 41.5, 36.9, 29.7, 28.3, 25.5, 22.4, 20.8, 18.3. LRMS (ESI): Calculated for C₂₇H₃₀CINO₄+H: 468.2, found: 468.2.

1. A process for synthesizing compound E, or a salt or solvate thereof: 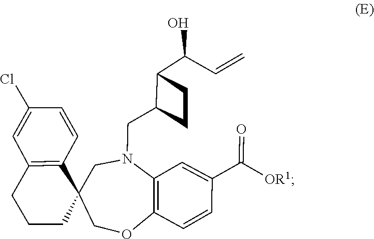
comprising admixing compound C, compound D, 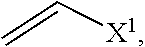
and Zn(X³)₂ in an organic solvent to form compound E: 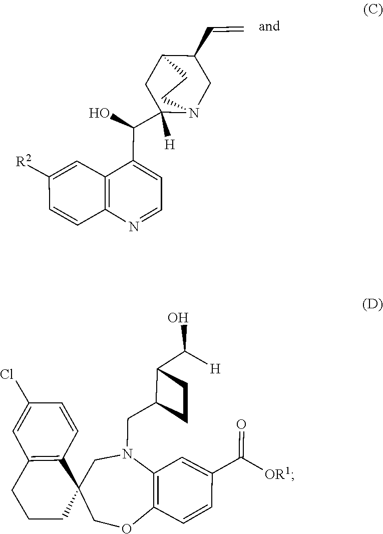
wherein
R¹ is C₁₋₆alkyl;
R² is H or C₁₋₃alkoxy;
X¹ is MgCl, MgBr, MgI, Li, CuLi, ZnX², In(I), In(X²)₂;
each X² independently is Cl, Br, or I; and
each X³ independently is Cl, Br, I, OTf, OTs, OAc, or acac.

2. The process of claim 1, wherein R¹ is methyl, ethyl, propyl, n-butyl, or tert-butyl.

3. The process of claim 2, wherein R¹ is methyl, ethyl, or tert-butyl.

4. The process of claim 1, wherein R² is H.

5. The process of claim 1, wherein R² is C₁₋₃alkoxy.

6. The process of claim 5, wherein R² is methoxy.

7. The process of claim 1, wherein X¹ is MgCl.

8. The process of claim 1, wherein X¹ is MgBr or MgI.

9. The process of claim 1, wherein X¹ is Li.

10. The process of claim 1, wherein X¹ is CuLi.

11. The process of claim 1, wherein X¹ is In(I) or In(X²)₂.

12. The process of claim 1, wherein X¹ is ZnCl or ZnBr.

13. The process of claim 1, wherein Zn(X³)₂ is ZnCl₂.

14. The process of claim 1, wherein Zn(X³)₂ is ZnBr₂.

15. The process of claim 1, wherein Zn(X³)₂ is ZnI₂.

16. The process of claim 1, wherein Zn(X³)₂ is Zn(OTf)₂ or Zn(OTs)₂.

17. The process of claim 1, wherein Zn(X³)₂ is Zn(OAc)₂ or Zn(acac)₂.

18. The process of claim 1, wherein the organic solvent is degassed prior to the admixing.

19. The process of claim 1, wherein the organic solvent comprises an ether solvent or acetonitrile.

20. The process of claim 19, wherein the organic solvent is selected from the group consisting of tetrahydrofuran (THF), 2-methyltetrahydrofuran (2-MeTHF), diethyl ether, acetonitrile, 1,2-dimethoxyethane (1,2-DME), methyl tert-butyl ether (MTBE), cyclopentyl methyl ether (CPME), and a combination thereof.

21. The process of claim 20, wherein the organic solvent is acetonitrile.

22. The process of claim 1, wherein the admixing is performed at a temperature of 10° C. to 35° C.

23. The process of claim 1, wherein the admixing comprises
(a) admixing compound C and Zn(X³)₂ in the organic solvent to form a suspension;
(b) adding 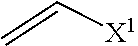
to the suspension to form a solution; and
(c) adding compound D to the solution to form compound E.

24. The process of claim 23, wherein the suspension of step (a) is cooled to a temperature of −15° C. to −5° C. prior to adding

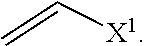

25. The process of claim 23, wherein 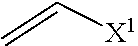
is added to the suspension as a solution in an ether solvent.

26. The process of claim 25, wherein the ether solvent is THF.

27. The process of claim 23, wherein 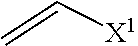
is added to the suspension at a temperature of −10° C. to 0° C.

28. The process of claim 23, wherein the solution of step (b) is brought to a temperature of 10° C. to 35° C. prior to adding compound D.

29. The process of claim 23, wherein compound D is added as a solution in an organic solvent selected from the group consisting of THF, 2-MeTHF, diethyl ether, acetonitrile, 1,2-DME, MTBE, CPME, and a combination thereof.

30. The process of claim 29, wherein the organic solvent comprises acetonitrile.

31. The process of claim 1, wherein compound D and 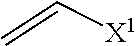
are present in a molar ratio of 1:2.5 to 1:4.5.

32. The process of claim 31, wherein the molar ratio of compound D to 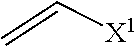
is 1:3.2.

33. The process of claim 1, wherein compound D and Zn(X³)₂ are present in a molar ratio of 1:2.5 to 1:4.0.

34. The process of claim 33, wherein the molar ratio of compound D to Zn(X³)₂ is 1:3.1.

35. The process of claim 1, wherein compound D and compound C are present in a molar ratio of 1:1 to 1:2.

36. The process of claim 35, wherein the molar ratio of compound D to compound C is 1:1.4.

37. The process of claim 1, wherein compound D is prepared by oxidizing compound B: 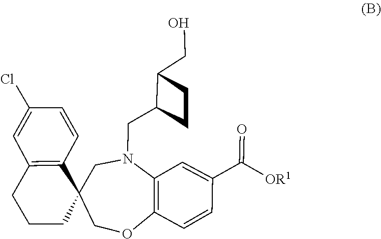
in the presence of an oxidizing agent and an organic solvent.

38. The process of claim 37, wherein the oxidizing occurs under an inert atmosphere.

39. The process of claim 37, wherein compound B is provided as a solution in an organic solvent selected from the group consisting of dimethyl sulfoxide (DMSO), dichloromethane (DCM), dimethylformamide (DMF), THF, 2-MeTHF, acetonitrile toluene, 1,2-DME, MTBE, 1,2-dichloroethane (DCE), chloroform, and a combination thereof.

40. The process of claim 39, wherein the organic solvent is DCM.

41. The process of claim 37, wherein the oxidizing agent is selected from the group consisting of oxalyl chloride, bleach, SO₃/pyridine, iodobenzenediacetate, trifluoroacetic anhydride, N-chlorosuccinimide (NCS), 2-iodooxybenzoic acid (IBX), N-methylmorpholine N-oxide (NMO), ceric ammonium nitrate (CAN), Dess-Martin periodinane, pyridinium chlorochromate (PCC), pyridinium dichromate (PDC), tetrapropylammonium perruthenate (TPAP)/NMO, NCS/dimethylsulfide, NCS/dodecyl sulfide, and a combination thereof.

42. The process of claim 41, wherein the oxidizing agent is oxalyl chloride.

43. The process of claim 37, wherein the oxidizing is performed in the presence of a base selected from the group consisting of triethylamine, diisopropylethanolamine, N-methylpyrrolidine, N-ethylpiperidine, pyridine, 2,2,6,6-tetramethylpiperidine (TMP), pempidine, 2,6-lutidine, and a combination thereof.

44. The process of claim 43, wherein the base is triethylamine.

45. The process of claim 37, wherein compound B and the oxidizing agent are present in a molar ratio of 1:1 to 1.3.

46. The process of claim 45, wherein the molar ratio of compound B to the oxidizing agent is 1:1.5.

47. The process of claim 43, wherein compound B and the base are present in a molar ratio of 1:3 to 1:10.

48. The process of claim 47, wherein the molar ratio of compound B to the base is 1:5.

49. The process of claim 37, wherein the oxidizing occurs in an organic solvent selected from the group consisting of dimethyl sulfoxide (DMSO), dichloromethane (DCM), dimethylformamide (DMF), THF, 2-MeTHF, acetonitrile, MTBE, 1,2-DME, toluene, DCE, CPME, and a combination thereof.

50. The process of claim 49, wherein the organic solvent is DMSO.

51. The process of claim 37, wherein the oxidizing occurs at a temperature of −80° C. to −20° C.

52. The process of claim 51, wherein the oxidizing occurs at a temperature of −40° C.

53. The process of claim 1, further comprising hydrolyzing compound E to form compound F: 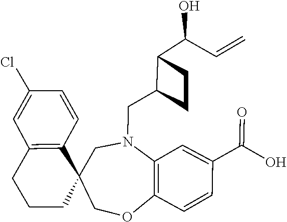
or a salt thereof.

54. The process of claim 53, wherein the hydrolyzing comprises:
admixing a solution of compound E in an organic solvent and a hydroxide base in water to form compound F.

55. The process of claim 54, wherein the hydroxide base is selected from the group consisting of NaOH, KOH, LiOH, potassium trimethylsilanolate (TMSOK), and a combination thereof.

56. The process of claim 54, wherein compound E and the hydroxide base are present in a molar ratio of 1:1 to 1:100.

57. The process of claim 56, wherein the molar ratio of compound E to the hydroxide base is 1:3.

58. The process of claim 54, wherein the organic solvent is selected from the group consisting of methanol, ethanol, propanol, isopropanol, butanol, THF, diethyl ether, acetone, acetonitrile, 2-MeTHF, sec-butanol, and a combination thereof.

59. The process of claim 58, wherein the organic solvent is ethanol.

60. The process of claim 54, wherein the hydrolyzing occurs at a temperature of 20° C. to 60° F.

61. The process of claim 53, wherein compound F is in salt form.

62. The process of claim 61, wherein the salt of compound F comprises an ammonium cation or an alkali metal cation.

63. The process of claim 62, wherein the ammonium cation is selected from the group consisting of benzylammonium, methylbenzylammonium, trimethylammonium, triethylammonium, morpholinium, pyridinium, piperidinium, picolinium, dicyclohexylammonium, protonated N,N′-dibenzylethylenediamine, 2-hydroxyethylammonium, bis-(2-hydroxyethyl)ammonium, tri-(2-hydroxyethyl)ammonium, protonated procaine, dibenzylpiperidium, dehydroabietylammonium, N,N′-bisdehydroabietylammonium, protonated glucamine, protonated N-methylglucamine, protonated collidine, protonated quinine, protonated quinoline, protonated lysine, protonated arginine, protonated 1,4-diazabicyclo[2.2.2]octane (DABCO), N,N-diisopropylethylammonium, and a combination thereof.

64. The process of claim 63, wherein the ammonium cation is

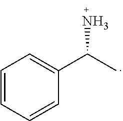

65. The process of claim 62, wherein the alkali metal cation is selected from the group consisting of lithium, sodium, potassium, and a combination thereof.

66. The process of claim 62, wherein the salt of compound F is prepared by admixing compound F, as its free acid form (compound F free acid), with an amine base or an alkali metal base in a nonpolar organic solvent to form the salt of compound F.

67. The process of claim 66, wherein compound F free acid and amine base or alkali metal base are present in a molar ratio of 1:1 to 1:2.

68. The process of claim 67, wherein the molar ratio of compound F free acid to amine base or alkali metal base is 1:1.2.

69. The process of claim 66, wherein the nonpolar organic solvent is selected from the group consisting of ethyl acetate, toluene, isopropyl acetate, MTBE, and a combination thereof.

70. The process of claim 69, wherein the nonpolar organic solvent is ethyl acetate.

71. The process of claim 66, wherein the admixing occurs at a temperature of 50° C. to 60° C.

72. The process of claim 61, wherein the admixing occurs in an inert atmosphere.

73. The process of claim 1, further comprising synthesizing compound A1 or a salt or solvate thereof using compound E:

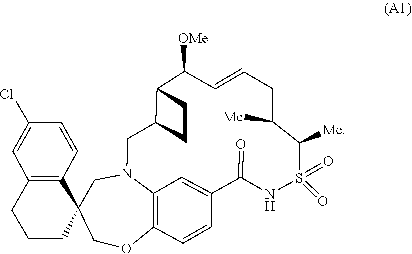

74. The process of claim 1, further comprising synthesizing compound A2 or a salt or solvate thereof using compound E: [chemical structure]
